(19) United States
(12) Patent Application Publication
(10) Pub. No.: US 2006/0189512 A1
(43) Pub. Date: Aug. 24, 2006

(54) Compositions containing botanical extracts rich in phlorizin and methods for using such compositions in blood glucose modification and to affect aging

(76) Inventor: Joel R. L. Ehrenkranz, Salt Lake City, UT (US)

Correspondence Address: MORRISS O'BRYANT COMPAGNI, P.C., 136 SOUTH MAIN STREET, SUITE 700, SALT LAKE CITY, UT, 84101, US

(21) Appl. No.: 11/336,629

(22) Filed: Jan. 20, 2006

Related U.S. Application Data
(60) Provisional application No. 60/645,796, filed on Jan. 21, 2005.

(51) Int. Cl. A61K 38/28; A61K 31/7048

(52) U.S. Cl. 514/3; 514/27

(57) ABSTRACT

Compositions are disclosed which contain therapeutically effective amounts of phlorizin extract for effecting modification of blood glucose and insulin, for facilitating weight loss, preventing weight gain and for providing beneficial effects in the aging process. Methods of treating animals with phlorizin extract compositions to treat the aforementioned conditions of the body are also disclosed.

### CROSS REFERENCE TO RELATED APPLICATIONS

This is a non-provisional application claiming priority to U.S. provisional application Ser. No. 60/645,796 filed Jan. 21, 2005.

### BACKGROUND

1. Field of the Invention

This invention relates to the modification of blood glucose and insulin levels in the treatment of certain diseases or medical conditions of the body, as well as the control of weight, aging and development of diseases. This invention specifically relates to the use and administration of botanical compositions rich in phlorizin in the modification of blood glucose and insulin, to facilitate weight loss and to provide beneficial effects in the aging process.

2. Statement of Related Art

Phlorizin, a dihydrochalcone, is a naturally-occurring product and dietary constituent found in a variety of fruit trees such as apple, and in fruit-bearing plants such as strawberry. Phlorizin has been shown to be present in the root, bark, shoots, leaves and fruit of certain fruit trees, and is also present in the plant parts and fruit of, for example, strawberry plants.

Phlorizin has been used in the past as a pharmaceutical tool for the evaluation of renal function and as a tool for investigation of glucose transport, as disclosed in U.S. Pat. No. 6,448,232. Phlorizin has also been demonstrated to be effective in improving insulin sensitivity by lowering blood sugar and has been suggested as useful in other blood glucose modulating methodologies.

### BRIEF DESCRIPTION OF THE INVENTION

In accordance with the present invention, methods for treating disease conditions, facilitating weight loss, preventing the development of disease or other undesirable conditions of the body and affecting aging in animals is mediated by modifying blood glucose and insulin levels through the use or administration of phlorizin extracts to animals. As used herein, “animals” or “patients” is intended to include both human and non-human animals. Phlorizin extracts are administered in amounts that effectively reduce glucose and insulin levels in the blood, and in effective amounts to control weight loss or prevent weight gain.

The phlorizin extract effectively modifies blood glucose and insulin levels, in accordance with the invention, by being administered in an effective or therapeutic amount or dosage, prior to the intake of food or a meal. By “effective amount”, “therapeutic amount” or “effective dose” is meant that amount of phlorizin extract sufficient to elicit the desired pharmacological or therapeutic effects, thus resulting in effective treatment, or prevention, of a disorder, disease or body condition. The phlorizin extract is administered within about 30 minutes to about 60 minutes prior to consumption of food or a meal.

The administration of phlorizin extracts is demonstrated to be effective in reducing or beneficially modifying blood glucose and insulin levels, to effect weight loss or prevent weight gain, and to have beneficial effects on the aging process and to increase longevity.

The phlorizin extracts may be derived from any source, particularly including but not limited to apple and cherry trees, apple fruit, pommice, peels, cider, and strawberry plants. The extracts may, in particular, be obtained from the twigs, bark, immature or mature fruits, or other aerial and subsurface parts of the tree or from fruit-bearing plants such as strawberry plants.

The phlorizin extracts used in accordance with the present invention comprise a standardized phlorizin content of from between 0.5% to 99%. A particularly suitable phlorizin extract of the present invention contains a standardized phlorizin content of about 20% to about 80%, and about 40% may be suitable for most administrations. The phlorizin extracts of the present invention may also contain other polyphenols including, for example, phloretin, catechins, chlorogenic acid, quercetin and polymers thereof.

The phlorizin extracts of the present invention have been demonstrated to have beneficial effects in producing glucosuria, decreasing postprandial rises in glucose and insulin levels, elimination of reactive hypoglycemia, control of stress hyperglycemia, weight loss and in preventing weight gain. Additionally, data show that there is a dose response relationship between the amount of phlorizin extract ingested and the amount of glucose excreted. Consequently, the more phlorizin extract one takes, the more glucose, calories, and weight one loses. The administration of phlorizin is also shown to have potentially beneficial anti-aging effects and to mimic mechanisms known to increase and activate increased longevity in animals.

### DETAILED DESCRIPTION OF THE INVENTION

The phlorizin extracts of the present invention may be derived from any source of phlorizin, including but not limited to fruit trees, the fruit of such trees and fruit-bearing plants, including the fruit of such plants. In particular, however, phlorizin extracts of the present invention may, most suitably, be derived from apple trees and strawberry plants. The phlorizin extract may be derived from twigs, bark, immature or mature fruits, or other aerial parts of the tree, from pommice, peels and cider, and from subsurface parts (e.g., roots) of the tree. Phlorizin may also be extracted from all parts of strawberry plants, including the fruit. The phlorizin extracts of the present invention may also contain other polyphenols including, for example, phloretin, catechins, chlorogenic acid, quercetin and polymers thereof. Such polyphenols may be extracted from the same source as the phlorizin.

The phlorizin extracts of the present invention may be produced by any means or method that produces the desired concentration of phlorizin. Exemplar methods of producing phlorizin extracts include water-based extraction of target materials by soaking parts of the tree (e.g., apple tree), such as twigs, bark and the fruit of the tree in water. Extraction may also be effected by use of ethanol or other suitable materials, as well as by supercritical CO 2 extraction. Such extraction methods are well-known in the art.

The phlorizin extracts may be administered in any desired form including orally, parenterally, by injection or by suppositories by known methods that are standard in the art. A preferred formulation for administration is as a dietary supplement or nutraceutical that is in tablet, capsule or powder form for oral administration. However, pharmaceutical compositions (wt. %) containing phlorizin extract as the active ingredient, along with a suitable carrier or vehicle, is within the scope of the invention. The means for forming such administrable forms are well-known in the art. The phlorizin extracts used in accordance with the present invention comprise a standardized phlorizin content of from between 0.5% to 99%. A particularly suitable phlorizin extract of the present invention contains a standardized phlorizin content of about 20% to about 80%, with a preferred content of about 40%.

The phlorizin extracts may, in certain forms, demonstrate limited or compromised bioavailability. Thus, phlorizin extracts of the present invention may be provided in encapsulated forms, such as complexing phlorizin with phosphotidylcholine, or delivering phlorizin in a solution/suspension with organic solvents such as polypropylene glycol, to increase bioavailability and extend the duration of the effectiveness of the product. Other methods of increasing bioavailability of the phlorzin extracts may be employed as required.

The administered dose of phrlorizin extract in accordance with the present invention may be from about 3 grams to about 30 grams per day. The daily dosage is administered in three separate doses comprising between about one gram to about 10 grams of phlorizin taken prior to the consumption of food or a meal. Each preprandial dosage may be taken from between about 30 minutes to an hour before consumption of a meal. The effective results of the intake of phlorizin extract are described more fully below.

The data in Table 1, immediately below, generally demonstrate that glucosuria increases and blood glucose levels decrease following consumption of a meal when phlorizin is administered prior to the meal on an empty stomach. Adult male subjects were provided either a placebo capsule or a capsule containing about 40% phlorizin extract in an amount of either 1 gm, 1.5 g, 2.0 g or 2.5 g. The capsules were taken orally sixty minutes prior to taking a meal. The subjects were fed a meal of 52 grams of sugar at time 0. Blood and urine glucose levels were measured at −60, 0, 30 and 60 minutes relative to the consumption of food. It can be seen from the test results that glucosuria increases and blood glucose decreases with higher dosages of phlorizin.

TABLE I

| | | blood | | |
|---|---|---|---|---|
| Dose | Time | glucose | Blood glu diff | Urine glucose |
| (gram) | (min) | (mg/dl) | (mg/dl) | (mg/dl) |
| 0 | −60 | 107 | 0 | 0 |
| 0 | 0 | 100 | −7 | 0 |
| 0 | 30 | 165 | 58 | 0 |
| 0 | 60 | 120 | 13 | 0 |
| 0 | 90 | | | 0 |
| 1 | −60 | 96 | 0 | 0 |
| 1 | 0 | 111 | 15 | 100 |
| 1 | 30 | 152 | 56 | 250 |
| 1 | 60 | 113 | 17 | 250 |
| 1 | 90 | | | 0 |
| 1.5 | −60 | 95 | 0 | 0 |
| 1.5 | 0 | 103 | 8 | 250 |
| 1.5 | 30 | 137 | 42 | 500 |
| 1.5 | 60 | 141 | 46 | 500 |
| 1.5 | 90 | | | 0 |
| 2 | −60 | 110 | 0 | 0 |
| 2 | 0 | 110 | 0 | 250 |
| 2 | 30 | 140 | 30 | 500 |
| 2 | 60 | 138 | 28 | 500 |
| 2 | 90 | | | 0 |
| 2.5 | −60 | 105 | 0 | 0 |
| 2.5 | 0 | 95 | −10 | 1000 |
| 2.5 | 30 | 133 | 28 | 1000 |
| 2.5 | 60 | 118 | 13 | 500 |
| 2.5 | 90 | 85 | −20 | 250 |
| 2.5 | 120 | 90 | −15 | 0 |

Graph A, immediately below, further demonstrates the relative effects of an administration of varying dosages, from 0.0 grams to 2.5 grams, of a phlorizin extract standardized to about a 40% content on blood glucose levels following consumption of 52 grams of carbohydrate sixty minutes after oral ingestion of the phlorizin. It can be seen that blood glucose levels are appreciably lowered with an increased dosage of phlorizin.

[image]

The data of TABLE II, immediately below, further demonstrate the modification of blood glucose levels affected by administration of 1.5 grams of 40% phlorizin extract administered sixty minutes prior to the consumption of a 100 gram carbohydrate meal (600 Kcal).

TABLE II

| | | Blood | Blood glucose |
|---|---|---|---|
| Capsules | Time (min) | glucose (mg/dl) | change (% from baseline) |
| Placebo | −60 | 111 | 0.00% |
| Ate lunch | 0 | 107 | −4.00% |
| | 30 | 134 | 23.00% |
| | 60 | 142 | 31.00% |
| | 90 | 117 | 6.00% |
| | 120 | 130 | 19.00% |
| Phlorizin | −60 | 97 | 0.00% |
| Ate lunch | 0 | 79 | −18.00% |
| | 30 | 113 | 16.00% |
| | 60 | 118 | 21.00% |
| | 90 | 123 | 26.00% |
| | 120 | 99 | 2.00% |

B, immediately below, further demonstrates these changes.

[image]

Table III, immediately following, displays the effects of phlorizin on the postprandial rise in glucose and insulin levels. The subjects were nine healthy adult normal volunteers. Each subject served as his or her own control. Subjects took a placebo pill one hour prior to carbohydrate ingestion on one day and 1.5 grams of phlorizin on another. The phlorizin extract content was standardized to about 40%. Glucose and insulin levels were measured at frequent intervals. The data in this table show that phlorizin, compared to placebo, produced a statistically significant blunting of the rise in serum glucose and insulin levels following carbohydrate ingestion.

TABLE III

| | Phlorizin | Placebo | |
|---|---|---|---|
| | 60 min. - baseline | 60 min. - baseline | p |
| Glucose (mg/dl) | | | |
| N | 9 | 9 | 0.016 |
| Mean | 31.11 | 50.78 | |
| Standard deviation | 26.41 | 28.77 | |
| Median | 26.0 | 52.0 | |
| Range | (1-82) | (12-90) | |
| Insulin (mIU/ml) | | | |
| N | 9 | 9 | 0.045 |
| Mean | 29.50 | 34.25 | |
| Standard deviation | 17.29 | 35.33 | |
| Median | 23.0 | 39.0 | |
| Range | (14-67) | (13-118) | |

The administration of phlorizin from an apple extract has further been shown to have beneficial effects in weight loss. As an example, a study group was treated with phlorizin for a period of fourteen to thirty-three days to determine the effect of the extract on weight loss. The study group consisted of ten adults, comprising both males and females, aged 24 to 71. The initial weights of the subjects ranged from between 145 and 307 pounds. The subjects were told not to change their routine eating or exercise habits for the period of the study. The subjects were administered 1.5 grams of 40% phlorizin extract 30 minutes before taking a meal. The subjects had not eaten for at least 4 hours prior to consuming the meal so that the phlorizin was administered on an empty stomach.

The subjects were weighed periodically over a period of from seven to thirty-three days. The subjects who were weighed at seven days had an average weight loss of 8 lbs. Weight loss for all of the subjects increased over the subsequent 19 days. The data from this study is shown in Table IV below.

TABLE IV

| Day | TN | WG | LM | YT | AE | JE | GW | VV | GG | CF |
|---|---|---|---|---|---|---|---|---|---|---|
| 0 | 210 | 252 | 210 | 307 | 242 | 202 | 145 | 185 | 180 | 161 |
| 4 | | | | | 241 | | | | | 160 |
| 6 | | | | 297 | | | | | | 159 |
| 7 | | | | | | 198 | | | | |
| 8 | | | | | 240 | | | | | |
| 9 | | | | 290 | | | | | | |
| 11 | | | | | 236.5 | | | | | 159 |
| 14 | 198 | | | 287 | | 196.6 | 140 | 177 | | |
| 15 | | | | | 235 | | | | | |
| 17 | | | | 285.5 | | | | | | 158 |
| 19 | | | | | 233.5 | | | | | |
| 21 | | | | | | 195 | | | | |
| 24 | | | | | 231 | | | | | 157 |
| 26 | | 237 | 205 | | | | | | | |
| 27 | | | | | | 193.8 | | | 175 | 155 |
| 28 | | | | | | | | | | |
| 33 | | | | 284 | | | | | | 153 |

The beneficial effect of the phlorizin extracts of the present invention on modification of glucose transport, blood and urine glucose levels, blood insulin levels, and weight loss are enhanced by the synergistic effects of other elements administered with phlorizin extract of the present invention. Such synergistic effects are potentially mediated by quercetin, phloretin, epicatechin, catechins, chlorogenic acid, their polymers and other elements extracted from fruit trees or plants.

Additionally, there is a demonstrated synergistic effect on the modification of glucose transport, blood and urine glucose levels, blood insulin levels and weight loss in individuals with type 1 or type 2 diabetes when the phlorizin extract of the present invention is administered with diabetes-related pharmaceuticals including, but not limited to, insulin, insulin secretogogues, insulin sensitizers (both thiazolidinediones and metformin), alpha-glucosidase inhibitors, or with other plant-based extracts with hypoglycemic properties, or both plant-based extracts and diabetes related pharmaceuticals. The following Examples are illustrative of these effects.

### EXAMPLE I

A thirty-one year old female with a fifteen year history of type 2 diabetes mellitus, who was not taking any anti-diabetic medications, was administered from between 1 and 5 grams of phlorizin extract, standardized to a content of about 40%, thirty minutes before taking a meal. The test results were as follows:

| | | After taking 3 | Three months |
|---|---|---|---|
| | | grams of apple | after |
| | | extract three | discontinuing |
| | Prior to starting | times a day | apple |
| | apple extract | before meals | extract |
| Fasting glucose | 160 mg/dl | 146 | 155 |
| Fasting insulin | 43 microIU/ml | 44 | 64 |
| Hemoglobin A1C | 8.1% | 7.3 | 7.6 |
| Weight | 276 lbs | 266 | 268 |

### EXAMPLE II

A 71 year old male with a seventeen year history of diabetes mellitus, who was taking prescribed insulin for the condition, was administered from between 1 and 5 grams of phlorizin extract, standardized to a content of 40%, thirty minutes before taking a meal. The test results were as follows:

| | | After taking 3 gm | |
|---|---|---|---|
| | Prior to | of apple extract | Three months after |
| | starting | three times a day | discontinuing apple |
| | apple extract | before meals | extract |
| Fasting glucose | 342 | 327 | 395 |
| Fasting insulin | n/a | n/a | n/a |
| Hemoglobin A1C | 10.8 | 10.2 | 12.2 |
| weight | 244 | 234 | 238 |

### EXAMPLE III

A 69 year old male with a five year history of type 2 diabetes, and who was on a prescribed regimen of metformin for the condition, was administered from between 1 and 5 grams of phlorizin extract, standardized to a content of 40%, thirty minutes before taking a meal. The test results were as follows:

| | | After taking 3 gm | |
|---|---|---|---|
| | Prior to | of apple extract | One month after |
| | starting | three times a day | discontinuing apple |
| | apple extract | before meals | extract |
| Fasting glucose | 138 | 140 | 143 |
| Fasting insulin | n/a | n/a | n/a |
| Hemoglobin A1C | 6.5 | 6.3 | n/a |
| weight | 246.5 | 236.5 | 246.5 |

Examples of plant-based substances and/or extracts having hypoglycemic properties, and which provide synergistic effect in combination with the phlorizin extract of the present invention to beneficially modify glucose transport, blood and urine glucose levels, blood insulin levels and weight loss include the following: Abroma augusta, Abutilon lignosum, Abutilon trisulcatum, Acacia Arabica, Acacia catechu seed, Acacia melanoxylon, Acacia retinodes, Acacia suma seed, Achyranthes aspera, Acosmium panamense bark, Acourtia thurberi, Acrocomia mexicana, Aegle marmelos, Agaricus bisporus, Agaricus blazei, Agarista mexicana, Agastache mexicana, Agave atrovirens, Agave lechequilla, Agave salmiana, Ageratina petiolaris, Ageratum conyzoides, Agrimonia eupatpria, Ajuga iva, Albizzia odoratissima seed, Alliona choisyi, Allium cepa, Allium sativum, Alloispermum integrifolium, Aloe barbadensis, Aloe vera, Alpinia officinarum, Ambrosia artemisiifolia, American ginseng leaf, Ammi visnaga, Amor seco, Anacardium occidentale, Anamu, Ananas comosus, Andiroba, Andrographis paniculata, Anethum graveolens, Annona cherimola, Annona glabra, Annona muricata, Anoectochilus formosanus, Apium graveolens, Aplidium conicum, Apodanthera buraeavi, Aporocactus flagelliformis, Arachis hypogaea, Arceuthobium vaginatum, Arctostaphylos pungens, Argemone mexicana, Argemone ochroleuca, Argemone platyceras, Argyria cuneater, Argyria speciosa, Aristolochia asclepiadifolia, Aristolochia malacophylla, Aristolochia sericea, Artemisia absinthium, Artemisia herba alba, Artemisia ludoviciana, Artemisia pallens wall, Artemisia santonicum, Artemisia vulgaris, Asclepias linaria, Asparagus racemosus, Asteracantha longifolia, Astragalus membranaceus, Atriplex halimus, Avena sativa, Averrhoa bilimbi, Azadirachta indica leaf, Baikal skullcap, Balanites aegyptiaca fruits, Bambusa dendrocalamus, Banaba, Barosma betulina, Bauhinia candicans, Bauhinia divaricata, Bauhinia forficata leaves, Bauhinia retusa seed, Ba-wei-die-huang-wan, Begonia heracleifolia, Berberine, Berberis moranensis, Bergenia ligulata, Beta vulgaris, Bidens aurea, Bidens leucantha, Bidens odorata, Bidens pilosa, Bignonia tuira, Black pepper, Bocconia arborea, Bombyx mori, Bouvardia ternifolia, Brassica nigra, Brassica oleracea var. gongylodes, Brazilian peppertree, Brickellia cavanillesii, Brickellia squarrosa, Brickellia veronicaefolia, Bridelia ferruginea, Brosium alicastrum, Bryonia alba, Buchnera pusilla, Buckwheat, Buddleia stachyoides, Buddleja Americana, Buddleja cordata, Buphorbia prostrate, Bursera simaruba, Byrsonima crassifolia, Cacalia decompisita, Cacalia peltata, Caesalpinia bonducella, Cajueiro, Calamintha macrostema, Calamintha officinalis moench, Calea hypoleuca, Calea integrifolia, Calea zacatechichi, Calliandra anomala, Callicarpa acuminate, Camellia sinensis, Canafistula, Capparis deciduas, Capraria biflora, Capsicum annum, Caralluma attenuate, Caralluma edulis, Carica papaya, Carqueja, Carya, Carya illinoensis, Casearia esculenta, Casimiroa edulis, Cassia alata leaf extract, Cassia fistula, Cassia skinneri, Cassia tomentosa, Castela texana, Castela tortuosa, Castilleja mutis, Catechin-gallate, Catharanthus roseus, Catuaba, Cecropia obtusifolia, Cecropia peltata, Ceiba pentandra, Celosia argentea linn, Centaurea aspera, Centaurium brachycalyx, Centaurium calycosum, Centella asiatica, Chamaecrista hispidula, Chamaemelum nobile, Chanca piedra, Chenopodium glaucum, Chitosan, Choline esterase, Chromolaena bigelovii, Cinnamomum cassia, Cinnamomum verum, Cinnamon, Cinnamon bark, Cirsium mexacanum, Cirsium pascuarense, Cirsium rhaphilepis, Cissampelos pareira, Cissus sicyoides, Citrullus colocynthis, Citrus aurantifolia, Citrus limetta, Citrus sinensis, Clausena anisata (wiild) hook, Clavillia, Cleome droserifolia, Cnidoscolus aconitifolius, Cnidoscolus chayamansa, Cnidoscolus multilobus, Coccinia cordifolia, Coccinia indica, Coffea Arabica, Cogniauxia podoleana baillon, Coix lacryma-jobi, Combretum farinosum, Commelina communis, Convolvuls mycrophyllus, Convolvulus pluricaulis, Conyza filaginoides, Conyza gnaphalioides, Cordia elaeagnoides, Cordia tinifolia, Coriandrum sativum L., Corni fructus, Coronopus didymus, Costus mexicanus, Costus rubber, Costus spicatus, Crataegus mexicana, Crataegus pubescens, Crinum, Crotalaria acapulcensis, Croton draco, Croton torreyanus, Cryptostegia grandiflora, Cucurbita ficifolia, Cucurbita maxima, Cucurbita mexicana, Cuminum nigrum, Curcuma longa, Cuscuta jalapensis, Cyamopis tetragonoloba (Linn.), Cyathea fulva, Cyclocarya paliurus, Cynanchum atratum, Cynara scolymus, Cynodon dactylon, Daidzin, Datura metel (Linn.), Dauvus carota, Di huang, Diasulin, Die-huang-wan, Dioscorea dumetorum, Diospyros digyna, Dorstenia contrajerva, Duranta repens, Dyssodia micropoides, Echinacea purpurea, Elaphoglossum, Elettaria cardamomum, Eleutherococcus senticosus, Embauba, Emblica officinalis, Enicostemma littorale, Equisetum giganteum, Equisetum hyemale, Equisetum myriochaetum, Eriobotrya japonica, Erva tost\overline{a}o, Espinheira santa, Eucalyptus globules, Eugenia jambolana, Eupatorium, Euphorbia maculate, Euphorbia prostrate, Euphrasia officinalis, Eysenhardtia polystachya, F. religiosa L., Fangchinoline, Fedegoso, Ficus bengalensis, ficus glomerata, Ficus racemosa bark extract, Ficus verens, Foeniculum vulgare, Folium mori, Fouquieria splendens, Fraxinus alba, Fraxinus excelsior, French lilac, Fumaria parviflora, Galega officinalis, Ganoderma lucidum, Genistein, Gentiana olivieri, Gervao, Ginseng, Globularia alypum, Glycine max, Gongronema latifolium leaves, Gossypium birsutum, Graviola, Green tea polyphenols, Grewia asiatica, Guacatonga, Guaiacum coulteri, Guaiacum sanctum, Guar gum, Guarana, Guardiola angustifolia, Guardiola tulocarpus, Guava, Guazuma ulmifolia, Gymnema hirsute, Gymnema montanum, Gymnema sylvestre, Gymnema yunnanense extract, Gynostemma pentaphyllum, Gynura procumbens, Haemahtoxylon brasiletto, Hamamelis virginiana, Hamelia patens, Hamiltonia suaveolens, Haplopappus venetus, Harpagophytum procumbens, Hawthorn, Hechtia melanocarpa, Heterotheca inuloides, Hibiscus rosa-sinensis L., Hidalgoa ternate, Hintonia latiflora, Hippocratea excelsa, Holy basil leaves, Hypoxis hemerocallidea corm, Ibervillea sonorae, lpomoea aquatica, lpomoea batatas L., Ipomoea pescaprae, Ipomoea starts, Iporuru, Jambolan, Jatoba, Jatropha dioica, Jatropha elbae, Juliania adstringens, Jurubeba, Justicia spicigera, Kalanchoepinnata, Kalopanax pictus extract, Karwinskia humboldtiana, Kohleria, Konjac-mannan, Lagerstroemia speciosa, Larrea tridentate, Latium, Laurus nobilis, Lentinus edodes. Lepechinia caulescens, Lepidium sativum, Lepidium virginicum, Leucaena leucocephala (Lam.) de wit, Leucas lavandulaefolia, Leucophyllumtexanum, Ligusticum porteria, Ligustrum japonicum, Linum usitatissimum, Lodiocea sechellarum, Loeselia coccinea, Loeselia mexicana, Lonchocarpus cruentus, Lopezia racemosa, Lophocereus schoftii, Lupinus termis, Lycium barbarum, Lyophyllum decastes, Lysiloma acapulcense, Lythrum salicaria, Macela, Malmae depresa, Malvastrum coromandelianum, Manaca, Mangifera indica, Marche, Marrubium vulgare, Medicago sativa. Medicago sativa, Melothria pendula, Memecylon umbellatum, Mentha piperita, Mentha rotundifolia, Mentha suaveolens, Mimosa zygophylla, Mirabilis jalapa, Mitragyna inermis (wild), Momordica charantia, Momordica cymbalaria hook, Morinda lucida leave, Morus alba, Morus indica, Morus nigra, Mucuna pruriens, Muira puama, Mullaca, Mulungu, Musa paradisiacal, Musa sapientum, Mutamba, Myrcia uniflora, Myricetin, Myristica fragrans, Nasturtium officinale, Nelumbo nucifera rhizome, Nepeta cataria, Nescafe, Neurolaena lobata, Nigella sativa L., Nopalea cochenillifera, Nopalea inaperta, Ocimum basilicum, ocimum flavonoids orientin, Ocimum gratissimum, Ocimum sanctum leaf, Oldenlandia affinis, Olea europaea, Oleum origami, Olive leaf, Onosma echioides, Opuntia atropes, Opuntia ficus-indica (L.), Opuntia fulgida, Opuntia guilanchi, Opuntia imbricate, Opuntia lindheimeri englem, Opuntia megacantha, Opuntia streptacantha, Origanum majorana leaves, Origanum vulgare, Orthosiphon stamineus, Pachira aquatica, Pachycereus, Pachycereus pringlei, Packera candidissima, Paeonia lactiflora, Panax ginseng, Panax quinquelfolius, Pandanus odorus, Parathesis lenticellata, Parkinsonia aculeate, Parmentiera aculeate, Parthenium hysterophorus, Pau d'arco, Pavonia schiedeana, Pedra hume caa, Pelvetia babingtonnii de toni, Persea Americana, Petroselinum crispum, Phaseolus vulgaris, Phlebodium aureum, Phoradendron tomentosum, Phragmites australis, Phyllanthus sellowianus, Physalis coztomatl, Physalis philadelphica, Phytosterin, Picao preto, Picrorrhiza kurroa, Pimenta officinalis, Pimpinella anisum, Piper auritum, Piper hispidum, Piper nigrum, Piper sanctum, Piper schideanum, Pithecellobium dulce, Planta australis, Plantago major L., Plumbago scandens, Plumeria rubra, Polygonati officinalis rhizome, Polygonati rhizome, Polygonatum sibricum, Polygonum acre, Pomegranate flower, Populus alba, Portulaca oleracae, Potentilla fulgens L., Poterium spinosum, Prunus dulcis, Psacalium decompositum, Psacalium peltatum, Psychotria oleoides, Pterocarpus marsupium wood, Pterocarpus santalinus L., Puemus boldus, Pueraria thunbergiana, Puerarin, Punica granatum Linn., Quassia amara, Quercetin, Ramulus mori, Red wine polyphenols, Rehmannia glutinosa. Retama raetam, Rhazya stricta, Rhizomes of alpinia offinarum hance, Rhizomes of polygala senega, Rhodiola rosea, Rhus verniciflua, Rhynchelytrum repens (willd), Rosmarinus officinalis, Rubus fructicosus, Saccharomyces cerevisiae, Salacia oblonga wall, Salacia reticulate, Salvia officinalis, Samambaia, Sangre de grado, Sanguis draxonis, Sclerocarya birrea, Scoparia dulcis, Scutellaria baicalensis, Securigera securidaca L. seed, Semecarpus anacardium Linn., Sida cordifolia, Silybum marianum, Simarouba, Smallantus sonchifolius, Spathodea campanulata stem bark, Spergularia purpurea, Stephania tetrandra radix, Stevia rebaudiana, Steviol, Suma, Swertia chirayita, Syzigium aramaticum, Syzigium cumini seeds, Syzygium alternifolium (wt) walp, Syzygium cumini, Tamarindus indica, Taraxacum officinale, Tayuya, Telfaria occidentalis, Terminalia arjuna, Terminalia catappa Linn., Terminalia chebula, Terminalia pallida fruit, Tetrapleura tetraptera, Teucrium polium, Thunbergia laurifolia Linn., Tinospora cordifolia, Tinospora crispa, Trifolium alexandrinum, Trigonella foenum-graecum, Triterpenoid glycosides, Triticum repens, Tulasi, Turnera aphrodisiaca, Turnera diffusa, Uncaria, Uncaria tomentosa, Uraria Picta, Urtica dioica, Vanilla planifolia, Verbesina encelioides, Vernonia colorata leaves, Vicenin. Vigna angularis, Withania coagulans dunal, Withania somnifera, Yerba mate, Zanthoxylum armatum, Zingiber officinale, Zizyphus sativa, Zizyphus spina-christi, and Zygophyllum gaetulum.

Caloric restriction has repeatedly been shown to slow the aging process and to increase the life span of a spectrum of organisms ranging from yeast to primates. The mechanisms through which caloric restriction act have not been clearly delineated; however reduced levels of glucose and insulin are hallmarks of caloric restriction in both rodents and primates. Phlorizin has been shown to decrease glucose and insulin levels in a manner analogous to caloric restriction, hence the administration of phlorizin extract of the present invention represents a caloric restriction mimetic.

By decreasing glucose levels, phlorizin extract is able to decrease the formation of advanced glycosylation end products. Hemoglobin A1c, routinely used in the assessment of diabetes control, is an example of an advanced glycosylation end product. The data presented above in Examples I, II and III demonstrate that the phlorizin extract of the present invention decreases advanced glycosylation end product formation and concentration. Decreasing the amounts of advanced glycosylation end products is associated with decreased aging-related pathologies, such as diabetes mellitus, Alzheimers disease, atherosclerosis, renal disease, osteoarthritis, and osteoporosis. Thus, by lowering glucose levels, phlorizin extract blunts the development of age-related pathologies due to the formation of advanced glycosylation end products.

Another mechanism through which phlorizin extract retards the aging process is via the genetic control of DNA transcription. Studies in yeast, worms, fruit flies, and rodents implicate a family of genes, termed sirtuins, which act as histone deacetylase enzymes, to regulate DNA transcription. A number of observations demonstrate the critical role sirtuins play in the regulation of aging. For example, in yeast and fruitflies, caloric restriction increases sirtuin activity, and duplicating the sirtuin 2 gene increases lifespan in these species, while deleting the sirtuin 2 gene reverses the longevity effect of caloric restriction.

A cell-based fluorescence deacetylase assay, SIRT1 Fluorimetric Drug Discovery Kit, obtained from BIOMOL Research Laboratories, Inc., Plymouth Meeting, Pa. was used to measure the effects of various polyphenols in apple extract on histone deacetylase activity. The results obtained are shown in TABLE V which follows:

TABLE V

| | | Histone deacetylase |
|---|---|---|
| Polyphenol | Concentration | activity |
| Phlorizin | 100 micromolar | 1.36 × baseline |
| Phloretin | 500 micromolar | 1.94 × baseline |
| Catechins | 500 micromolar | 1.26-1.53 × baseline |
| Quercetin | 500 micromolar | 4.59 × baseline |

These results demonstrate that components of apple extract increase histone deacetylase activity in manner consistent with the known anti-aging effects of histone deacetylase induction.

The data illustrate that phlorizin extract decreases the rate of aging through three separate mechanisms: As a caloric restriction mimetic to decrease glucose and insulin levels; by decreasing the formation of advanced glycation end products; and through increasing histone deacetylase activity.

The phlorizin extract of the present invention provides beneficial effects in the modification of glucose transport, blood and urine glucose levels and blood insulin levels. The phlorizin extract is also effective in producing weight loss and in prevention of weight gain, and has beneficial effects in increasing longevity and producing anti-aging affects in animals. The foregoing description of the specific embodiments will so fully reveal the general nature of the invention that others can, by applying current knowledge, readily modify and/or adapt for various applications such specific embodiments without departing from the generic concept and therefore such adaptations are intended to be comprehended within the meaning and range of equivalents of the disclosed embodiments. It is to be understood that the phraseology or terminology employed herein is for the purpose of description only and not of limitation.

What is claimed is:

1. A composition for beneficially modifying blood and urine glucose levels and facilitating weight loss, comprising a therapeutic amount of from between about 0.5% to about 99% phlorizin extract.

2. The composition of claim 1 wherein said phlorizin extract concentration is about 20 to about 80%.

3. The composition of claim 2 wherein said phlorizin extract concentration is about 40%.

4. The composition of claim 1 wherein said phlorizin extract is derived from the fruit, twigs, bark and aerial parts of fruit trees, from the fruit and plant parts of strawberry plants, and combinations thereof.

5. The composition of claim 1 wherein said phlorizin extract is complexed to increase bioavailability.

6. The composition of claim 1 further comprising a plant-based substance having hypoglycemic properties.

7. The composition of claim 1 further comprising a pharmaceutical substance selected from the group comprising insulin, insulin sensitizers, insulin secretagogues and alpha-glucosidase inhibitors.

8. A dietary supplement for modifying blood and urine glucose levels, lowering postprandial insulin levels, facilitating weight loss and increasing lifespan in animals, comprising a daily dosage of from between 3 grams to 30 grams of phlorizion extract.

9. The dietary supplement of claim 8 wherein said daily dosage further comprises a unitary dosage of between one gram and ten grams of phlorizin extract formulated for administration in three daily doses.

10. A method of treating or effecting disease or other body conditions in an animal by modifying blood glucose and insulin levels through administration of compositions containing phlorizin extract standardized to a content of between about 0.5% and about 99%.

11. The method of claim 10 wherein said treating or effecting of disease or body conditions is the decreasing of blood and insulin glucose levels.

12. The method of claim 10 wherein said treatment or effecting of disease or body condition is the elimination of reactive hypoglycemia.

13. The method of claim 10 wherein said treating or effecting of disease or body conditions is the amelioration of stress hyperglycemia.

14. The method of claim 10 wherein said treating or effecting of disease or body conditions is the controlling of weight changes in an animal.

15. The method of claim 14 wherein said controlling of weight changes is the facilitation of weight loss.

16. The method of claim 15 wherein said method further comprises the administration of said phlorizin extract, which is standardized to a concentration of about 40%, in a daily dosage of from between about three grams to thirty grams.

17. The method of claim 14 wherein said controlling of weight changes is the prevention of weight gain.

18. The method of claim 10 wherein said treating or effecting of disease or body conditions is the prevention of development of diabetes in patients with glucose intolerance.

19. The method of claim 18 wherein said treatment further comprises the co-administration of diabetes-related pharmaceuticals selected from the group comprising insulin secretogogues, insulin sensitizers and alpha-glucosidase inhibitors

20. The method of claim 10 wherein said treating or effecting of disease or body conditions is the increasing of longevity and decreasing of onset or incidence of age-related diseases by decreasing glucose and insulin levels.

21. The method of claim 10 wherein said treating or effecting of disease or body conditions is the increasing of histone deacetylase activity.

22. The method of claim 10 wherein said treating or effecting of disease or body conditions is the formation of advanced glycosylation end products.

23. The method of claim 10 wherein said administration of compositions containing phlorizin extract further comprises administering said compositions from thirty minutes to one hour prior to consumption of food or a meal.

24. The method of claim 10 wherein said concentration of phlorizin extract in said composition is between about 20% to about 40%.

25. The method of claim 10 wherein said phlorizin extract is extracted from the fruit, twigs, bark and aerial parts of fruit trees, from the fruit and plant parts of strawberry plants, and combinations thereof.

26. The method of claim 15 where the controlling of weight loss is mediated by selection of increased phlorizin extract administration to effect selected increased weight loss.
